(19) United States
(12) Patent Application Publication
(10) Pub. No.: US 2026/0174688 A1
(43) Pub. Date: Jun. 25, 2026

(54) POLYAMINE-MODIFIED GELATIN-BASED HYDROGEL AND USE THEREOF

(71) Applicant: SHANGHAI REUNION MEDICAL CO., LTD., Shanghai (CN)

(72) Inventors: Zhen PAN, Shanghai (CN); Jinpeng HUANG, Shanghai (CN)

(21) Appl. No.: 18/837,229

(22) PCT Filed: Feb. 1, 2024
(86) PCT No.: PCT/CN2024/075105

§ 371 Date: Aug. 9, 2024

(30) Foreign Application Priority Data
Mar. 2, 2023 (CN) 202310193534.X

(51) Int. Cl. A61K 9/06; A61K 31/405; A61K 31/4745; A61K 31/704; A61K 47/22; A61K 47/34; A61K 47/42

(52) U.S. Cl. CPC A61K 9/06; A61K 31/405; A61K 31/4745; A61K 31/704; A61K 47/22; A61K 47/34; A61K 47/42

(57) ABSTRACT

Disclosed is a polyamine-modified gelatin-based hydrogel and use thereof, belonging to the technical field of pharmaceutical preparations. A polyamine-modified gelatin is prepared by polyamine chemical modification and then cross-linked with a polyaldehyde-polyethylene glycol (CHO-PEG) derivative to form a hydrogel. The polyamine groups on the modified gelatin construct a hydrophobic core region.

The present application claims priority to the Chinese Patent Application No. 202310193534.X, filed with the China National Intellectual Property Administration (CNIPA) on Mar. 2, 2023, and entitled “POLYAMINE-MODIFIED GELATIN-BASED HYDROGEL AND USE THEREOF”, which is incorporated herein by reference in its entirety.

### TECHNICAL FIELD

The present disclosure relates to the technical field of pharmaceutical preparations, in particular to the field under IPC classification number A61K9/00, and more specifically to a polyamine-modified gelatin-based hydrogel and use thereof.

### BACKGROUND

Gelatin is a natural polypeptide polymer derived from collagen extracted from animal tissue, which possesses excellent biocompatibility and biodegradability. Depending on the preparation method, gelatin is classified into type-A gelatin, type B gelatin, and enzymatic gelatin. At the same time, due to its gelling properties, gelatin has been widely used in the medical field.

Hydrogel has a three-dimensional network structure, and can maintain its structure integrity while swelling after absorbing a large amount of water. Gelatin hydrogel can serve as an excellent drug carrier, which can protect drug molecules from external environment to a large extent and achieve a sustained release of drugs. Generally, hydrogels are hydrophilic substances capable of encapsulating hydrophilic drugs. However, there are also problems such as poor compatibility between hydrogels and certain types of drugs, such as low drug loading efficiency for certain hydrophobic drugs, and poor sustained release effect. Accordingly, encapsulating sufficient amount of hydrophobic drugs in hydrogels and then effectively controlling their release to achieve long-term drug delivery is an urgent problem that needs to be solved.

In the prior art, patent CN109316442A disclosed a supramolecular hydrogel drug delivery system that can encapsulate hydrophobic drugs. However, the preparation method for the supramolecular hydrogel drug delivery system is relatively complex, it suffers from severe burst release, low drug release rate, and reaches a plateau in about 7 days, failing to achieve long-term sustained release.

### SUMMARY

In view of the defects of the prior art, the objective of the present disclosure is to provide a polyamine-modified gelatin-based hydrogel with the characteristics of effective loading and sustained release of hydrophobic drugs, high drug release rate, and controllable drug release rate.

Moreover, the present disclosure also aims to provide use of the polyamine-modified gelatin-based hydrogel.

To achieve the above objective, the present disclosure adopts the following technical solutions:

The present disclosure provides a polyamine-modified gelatin-based hydrogel, the preparation raw materials of the polyamine-modified gelatin-based hydrogel includes amine-modified gelatin and polyethylene glycol derivatives with aldehyde functional groups (CHO-PEG).

In some embodiments, the mass ratio of the amine-modified gelatin to the CHO-PEG derivative is in the range of 1:(1-4); in specific embodiments, it is 1:1 or 1:2.

In some embodiments, the amine-modified gelatin is diamine C₆₋₁₀ alkane-modified gelatin.

In some embodiments, a preparation method of the diamine C₆₋₁₀ alkane-modified gelatin includes the following steps:
S1, dissolving gelatin in water to obtain a gelatin solution;
S2, adding a diamine C₆₋₁₀ alkane into the gelatin solution and mixing evenly by stirring to obtain a mixed solution;
S3, adding a condensing agent into the mixed solution obtained in step S2 to allow a temperature-controlled reaction to obtain a reaction solution; and
S4, dialyzing the reaction solution obtained in step S3 and freeze-drying to obtain the diamine C₆₋₁₀ alkane-modified gelatin.

In some embodiments, the diamine C₆₋₁₀ alkane is selected from the group consisting of hexanediamine, heptanediamine, octanediamine, nonanediamine, and decanediamine.

In some embodiments, the gelatin is type B gelatin with a gel strength of greater than or equal to 180 g Bloom.

In some embodiments, a concentration of the gelatin solution is in the range of 80 mg/mL to 120 mg/mL, in specific embodiments, it is 100 mg/mL.

In some embodiments, a molar ratio of the diamine C₆₋₁₀ alkane to the condensing agent is in the range of (8-12): 1, in specific embodiments, it is 1:1.

In some embodiments, the condensing agent is 4-(4,6-dimethoxytriazine-2-yl)-4-methylmorpholine hydrochloride (DMTMM) with a CAS number of 3945-69-5.

In some embodiments, the temperature-controlled reaction in step S3 is conducted at 25° C. to 37° C.

In some embodiments, the temperature-controlled reaction in step S3 is conducted for 36 h to 60 h, in specific embodiments, it is conducted for 48 h.

In some embodiments, the specific dialysis process in step S4 is as follows: dialyzing with a 1,000 Da dialysis bag in deionized water for 36 h to 60 h; in specific embodiments, dialyzing for 48 h.

In some embodiments, the 1,000 Da dialysis bag is a MWCO 1,000 Da dialysis bag.

In the present disclosure, diaminealkane is used to modify gelatin, and the diaminealkane is grafted onto gelatin through amide bonds, thereby increasing the amine content of gelatin. The polyamine-modified gelatin is cross-linked with the CHO-PEG derivative to form a dense and stable gel; polyamine groups are grafted on gelatin through hydrophobic alkane chain, forming a hydrophobic core region within the gel, thereby enabling the loading and sustained release of hydrophobic drugs. At the same time, the hydrophobic regions formed by the modified gelatin within the gel can stably retain hydrophobic drugs in the hydrogel system of the present disclosure, minimizing burst release.

The inventors have also creatively discovered that the drug release rate of the final hydrogel can be controlled by modifying gelatin with different diaminealkanes. When hexanediamine with a shorter alkane chain length is used, its hydrophobicity is weaker and its affinity for hydrophobic drugs is relatively weaker. Therefore, the drug retention effect in the hydrogel is slightly weaker, resulting in faster release rate. When gelatin is modified using decanediamine with a longer alkane chain length, its hydrophobicity is stronger, allowing the hydrophobic drug to be retained in the hydrogel for a longer time. This can achieve a slower and longer-term release, thereby minimizing burst release to a higher extend for most drugs and providing a longer-term therapeutic effect. This creative discovery allows the hydrogel of the present disclosure to be compatible with different drug properties and meet various complex clinical needs in actual use.

In some embodiments, the CHO-PEG derivative is a benzaldehyde-terminated star-shaped multi-arm PEG, with the number of arms of the benzaldehyde-terminated star-shaped multi-arm PEG ranging from 4 to 8, in specific embodiments, the number of arms is 4, 6, or 8.

In some embodiments, the benzaldehyde-terminated star-shaped multi-arm PEG is purchased from Beijing JenKem Technology Co., Ltd.

In the present disclosure, benzaldehyde-terminated star-shaped multi-arm PEG is selected to chemically crosslink with amine-containing modified gelatin to form a hydrogel; the gelation conditions are simple, requiring neither the action of a photoinitiator nor additional condition control. Moreover, not using the photoinitiator can also prevent some drugs, such as epirubicin, from participating in the photocrosslinking reactions and being chemically coupled to the main chain of the hydrogel, which can result in the drug not being released and thus having low drug bioavailability. The benzaldehyde-terminated star-shaped multi-arm PEG can fully cross-link with the polyamine-modified gelatin, resulting in a hydrogel with better stability and slower degradation rate, which is completely degraded after more than 42 days, providing a basis for achieving long-term sustained release.

Moreover, the present disclosure aims to provide use of the polyamine-modified gelatin-based hydrogel in encapsulation and sustained release of a drug.

In some embodiments, the encapsulation of the drug includes the following steps: dissolving the drug in the CHO-PEG derivative solution, and then mixing an obtained mixture with the amine-modified gelatin solution to obtain a hydrogel.

In some embodiments, the CHO-PEG derivative in the CHO-PEG derivative solution has a mass concentration in the range of 10% to 30%, in specific embodiments, it is 20%.

In some embodiments, the amine-modified gelatin in the amine-modified gelatin solution has a mass concentration in the range of 5% to 20%, in specific embodiments, it is 10% or 20%.

In some embodiments, a concentration of the drug in the mixture obtained by dissolving the drug in the CHO-PEG derivative solution is in the range of 0.5 mg/mL to 1.5 mg/mL, in specific embodiments, it is 1 mg/mL.

In some embodiments, the drug is a hydrophobic drug selected from the group consisting of epirubicin, indomethacin, and irinotecan.

In some embodiments, a solvent of the amine-modified gelatin solution is a 0.03 M borax buffer.

In some embodiments, a solvent of the CHO-PEG derivative solution is a 0.2 M sodium phosphate monobasic buffer.

In some embodiments, the hydrogel is obtained by mixing the CHO-PEG derivative solution containing the drug and the amine-modified gelatin solution at a volume ratio of 1:1.

Compared with the prior art, the present application has the following beneficial effects:
1. In the present disclosure, amine-modified gelatin is used to prepare the hydrogel, which provides excellent loading and sustained-release effects for the hydrophobic drug.
2. In the present disclosure, the hydrophobic core region constructed by amine alkane groups on the gelatin shows desirable affinity for hydrophobic drugs, effectively preventing the burst release of drugs.
3. In the present disclosure, the relationship between the type of diamine alkane used to modify gelatin and the drug release rate of hydrogel is discovered for the first time. The selection of diamine alkane types enables the control of the release rate of different drugs, thus meeting various complex clinical demands.
4. In the present disclosure, the CHO-PEG derivative is used to chemically cross-link with the modified gelatin to form the hydrogel, thereby preventing the drug from being coupled to the main chain of the hydrogel and unable to be released.
5. In the present disclosure, the CHO-PEG derivative and the polyamine-modified gelatin are fully cross-linked, and the formed hydrogel has the characteristics of desirable stability and slow degradation rate, which are conducive to achieving long-term sustained release.

### BRIEF DESCRIPTION OF THE DRAWINGS

FIG. 1 shows drug release curves of the drug-loaded hydrogels in Examples 1 to 3; and

FIG. 2 shows drug release curves of the drug-loaded hydrogels in Examples 4 to 6.

### DETAILED DESCRIPTION OF THE EMBODIMENTS

### Example 1

This example provided a polyamine-modified gelatin-based hydrogel, the preparation raw materials of the polyamine-modified gelatin-based hydrogel included amine-modified gelatin and CHO-PEG derivative; where
the amine-modified gelatin and the CHO-PEG derivative were at the mass ratio of 1:1;
the amine-modified gelatin was hexanediamine-modified gelatin, which was prepared by the following steps:
S1, 5.0 g of gelatin (type B, gel strength≥180 g Bloom) was fully dissolved in 50 mL of deionized water in the 37° C. water bath to obtain a gelatin solution with the concentration of 100 mg/mL.
S2, 11.62 g of hexanediamine was added to the gelatin solution obtained in S1, and stirred in the 37° C. water bath for 30 min.
S3, 2.7672 g of 4-(4,6-dimethoxytriazine-2-yl)-4-methylmorpholine hydrochloride (DMTMM) was added to the stirred solution in S2 and reacted in the water bath at 37° C. for 48 h.
S4, the reaction solution obtained in S3 was placed in MWCO 1,000 Da dialysis bag, dialyzed in deionized water for 48 h, freeze-dried, and the obtained lyophilizer was stored at 4° C.

The CHO-PEG derivative was benzaldehyde-terminated star-shaped 4-arm PEG purchased from Beijing JenKem Technology Co., Ltd.

Moreover, this example further aimed to provide use of the polyamine-modified gelatin-based hydrogel in encapsulation and sustained release of a drug; where
the drug was epirubicin hydrochloride; and
the encapsulation of the drug included the following steps: the benzaldehyde-terminated star-shaped 4-arm PEG was dissolved in 0.2 M sodium phosphate monobasic buffer to obtain a CHO-PEG derivative solution with the mass concentration of 20%; the epirubicin hydrochloride was dissolved in the benzaldehyde-terminated star-shaped 4-arm PEG solution to make the drug loading concentration of 1 mg/mL; the hexanediamine-modified gelatin was dissolved in 0.03 M borax buffer to obtain a hexanediamine-modified gelatin solution with the mass concentration of 10%; the drug-loaded benzaldehyde-terminated star-shaped 4-arm PEG solution and the hexanediamine-modified gelatin solution were mixed in equal volumes to form a hydrogel.

### Example 2

This example provided a polyamine-modified gelatin-based hydrogel and use thereof, wherein the implementation method was the same as that in Example 1, except that the drug was indomethacin.

### Example 3

This example provided a polyamine-modified gelatin-based hydrogel and use thereof, wherein the implementation method was the same as that in Example 1, except that the drug was irinotecan.

### Example 4

This example provided a polyamine-modified gelatin-based hydrogel, the preparation raw materials of the polyamine-modified gelatin-based hydrogel included amine-modified gelatin and CHO-PEG derivative; where
the amine-modified gelatin and the CHO-PEG derivative were at the mass ratio of 1:2;
the amine-modified gelatin was decanediamine-modified gelatin, which was prepared by the following steps:
S1, 1.5 g of gelatin (type B, gel strength≥180 g Bloom) was fully dissolved in 15 mL of deionized water in the 37° C. water bath to obtain a gelatin solution with the concentration of 100 mg/mL;
S2, 5.2 g of decanediamine was fully dissolved in 15 mL of ethanol under stirring to obtain a 2 M decanediamine ethanol solution; the decanediamine ethanol solution was added to the gelatin solution obtained in S1 under the 37° C. water bath and stirred to mix evenly.
S3, 0.8302 g of 4-(4,6-dimethoxytriazine-2-yl)-4-methylmorpholine hydrochloride (DMTMM) was added to the stirred solution in S2 and reacted in a water bath at 37° C. for 48 h.
S4, the reaction solution obtained in S3 was placed in MWCO 1,000 Da dialysis bag, dialyzed in deionized water for 48 h, freeze-dried, and the obtained lyophilizer was stored at 4° C.

The CHO-PEG derivative was benzaldehyde-terminated star-shaped 4-arm PEG purchased from Beijing JenKem Technology Co., Ltd.

Moreover, this example further aimed to provide use of the polyamine-modified gelatin-based hydrogel in encapsulation and sustained release of a drug; where
the drug was epirubicin hydrochloride; and
the encapsulation of the drug included the following steps: the benzaldehyde-terminated star-shaped 4-arm PEG was dissolved in 0.2 M sodium phosphate monobasic buffer to obtain a CHO-PEG derivative solution with the mass concentration of 20%; the epirubicin hydrochloride was dissolved in the benzaldehyde-terminated star-shaped multi-arm PEG solution to make the drug loading concentration of 1 mg/mL; the decanediamine-modified gelatin was dissolved in 0.03 M borax buffer to obtain a decanediamine-modified gelatin solution with the mass concentration of 10%; the drug-loaded benzaldehyde-terminated star-shaped 4-arm PEG solution and the decanediamine-modified gelatin solution were mixed in equal volumes to form a hydrogel.

### Example 5

This example provided a polyamine-modified gelatin-based hydrogel and use thereof, wherein the implementation method was the same as that in Example 4, except that the drug was indomethacin.

### Example 6

This example provided a polyamine-modified gelatin-based hydrogel and use thereof, wherein the implementation method was the same as that in Example 4, except that the drug was irinotecan.

### Comparative Example 1

This comparative example provided a PEGDA drug-loaded gel, wherein the preparation method was as follows:
1.1 g of PEGDA (CAS: 26570-48-9, purchased from Beijing Jenkem Technology Co., Ltd.) was dissolved in 4.4 g of PBS solution to obtain a 20% PEGDA solution, and added with the drug epirubicin hydrochloride to make the concentration of 1 mg/mL; and
the photoinitiator solution with the mass fraction of 0.1%, 2-hydroxy-4′-(2-hydroxyethoxy)-2-methylpropiophenone (CAS No.: 106797-53-9, purchased from Shanghai yuanye Bio-Technology Co., Ltd), was prepared. Equal volume of PEGDA drug-loaded solution and photoinitiator solution were mixed evenly; the resulting mixed solution was placed under a 52 W ultraviolet lamp to allow ultraviolet irradiation for 10 s to form the drug-loaded gel.

### Comparative Example 2

This comparative example provided a PEGDA drug-loaded gel, wherein its implementation method was the same as that of Comparative Example 1, except that the drug was indomethacin.

### Performance Test

### Drug Release Performance Test

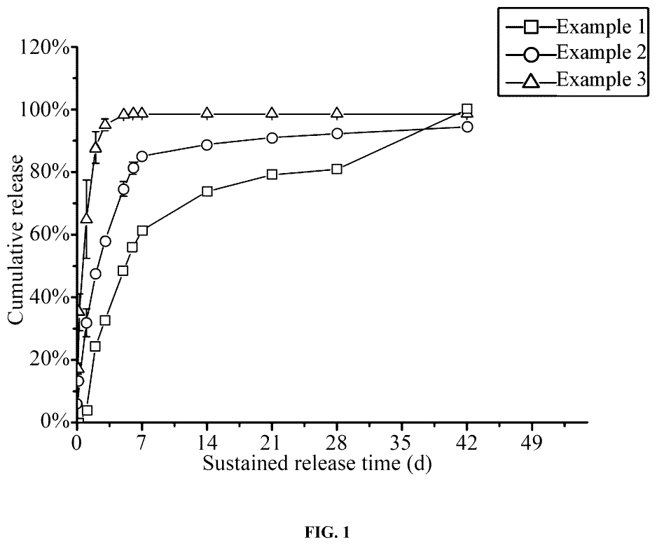

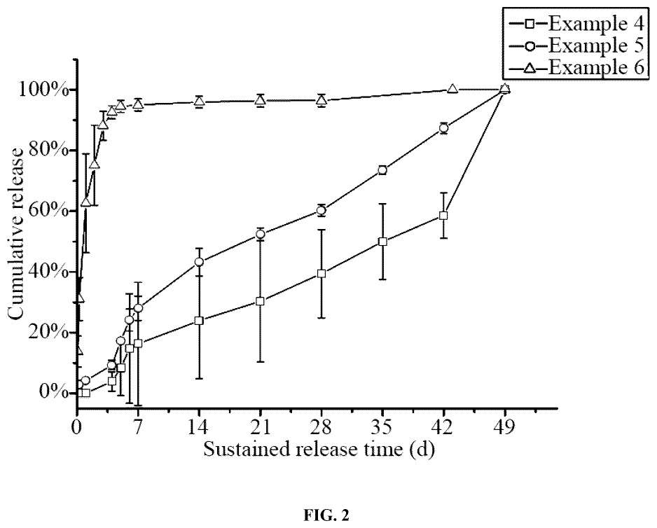

0.5 g of the drug-loaded gels obtained in Examples 1 to 6 and Comparative Examples 1 to 2 were respectively taken and placed in 5 mL of PBS solution for drug dissolution experiment, and the obtained mixtures were placed in a 37° C. water bath shaker. All extracts were taken out to measure the drug concentration at 2 h, 6 h, 24 h, 48 h, 72 h, 96 h, 120 h, 144 h, 168 h, 14 d, 21 d, 28 d, 35 d, 42 d, and 49 d, while 5 mL of blank PBS solution was supplemented; each group of experiments was repeated three times, and the test results were averaged to calculate the drug release rate, and the drug release curve of the drug-loaded gel was drawn (FIGS. 1-2).

The drug release rate data of Examples 1-6 and Comparative Examples 1 and 2 were shown in Tables 1-2. When the gel was completely degraded, the drug release rate was represented as 100%:

TABLE 1

| | Sustained release time | | | | | | | | | |
|---|---|---|---|---|---|---|---|---|---|---|
| drug loading type | Example | 2 h | 6 h | 24 h | 48 h | 72 h | 96 h | 120 h | 144 h | 168 h |
| PEGDA- | Comparative | 0 | 0 | 0 | 0 | 0 | 0 | 0 | — | — |
| epirubicin | Example 1 | | | | | | | | | |
| PEGDA- | Comparative | 15.08% | 24.50% | 36.39% | 42.06% | 46.16% | 49.49% | 51.75% | — | — |
| indomethacin | Example 2 | | | | | | | | | |
| Hexanediamine- | Example 1 | 0 | 0 | 3.91% | 24.33% | 32.67% | — | 48.41% | 55.73% | 61.40% |
| epirubicin | | | | | | | | | | |
| Hexanediamine- | Example 2 | 5.91% | 13.27% | 31.84% | 47.28% | 58.11% | — | 74.62% | 81.25% | 85.05% |
| indomethacin | | | | | | | | | | |
| Hexanediamine- | Example 3 | 17.23% | 35.21% | 64.94% | 87.86% | 95.07% | — | 98.22% | 98.49% | 98.49% |
| irinotecan | | | | | | | | | | |
| Decanediamine- | Example 4 | 0 | 0 | 0 | — | — | 3.90% | 8.28% | 14.75% | 16.32% |
| epirubicin | | | | | | | | | | |
| Decanediamine- | Example 5 | 1.68% | 2.95% | 4.20% | — | — | 9.32% | 17.23% | 24.11% | 28.01% |
| indomethacin | | | | | | | | | | |
| Decanediamine- | Example 6 | 13.78% | 31.06% | 62.63% | 75.15% | 88.13% | 92.58% | 94.49% | — | 95.01% |
| irinotecan | | | | | | | | | | |

TABLE 2

| | Sustained release time | | | | | | |
|---|---|---|---|---|---|---|---|
| drug loading type | Example | 14 d | 21 d | 28 d | 35 d | 42 d | 49 d |
| Hexanediamine-epirubicin | Example 1 | 73.80% | 79.13% | 80.84% | — | 100% | — |
| Hexanediamine-indomethacin | Example 2 | 88.81% | 91.02% | 92.27% | — | 94.41% | 100% |
| Hexanediamine-irinotecan | Example 3 | 98.49% | 98.49% | 98.49% | — | 98.49% | 100% |
| Decanediamine-epirubicin | Example 4 | 23.95% | 30.38% | 39.40% | 49.96% | 58.59% | 100% |
| Decanediamine-indomethacin | Example 5 | 43.20% | 52.35% | 60.25% | 73.54% | 87.37% | 100% |
| Decanediamine-irinotecan | Example 6 | 95.94% | 96.38% | 96.46% | — | 100% | — |

The objectives, technical solutions, and beneficial effects of the present disclosure are further described in detail in the above specific embodiments. It should be understood that the above described are merely specific embodiments of the present disclosure, and are not intended to limit the protection scope of the present disclosure. Any modification, equivalent replacement or improvement made within the spirit and principle of the present disclosure should fall within the protection scope of the present disclosure.

1. A polyamine-modified gelatin-based hydrogel, wherein preparation raw materials of the polyamine-modified gelatin-based hydrogel comprises amine-modified gelatin and an aldehyde-polyethylene glycol (CHO-PEG) derivative.

2. The polyamine-modified gelatin-based hydrogel according to claim 1, wherein a mass ratio of the amine-modified gelatin to the CHO-PEG derivative is in a range of 1:(1-4).

3. The polyamine-modified gelatin-based hydrogel according to claim 1, wherein the amine-modified gelatin is diamine C₆₋₁₀ alkane-modified gelatin.

4. The polyamine-modified gelatin-based hydrogel according to claim 3, wherein a preparation method of the diamine C₆₋₁₀ alkane-modified gelatin comprises the following steps:
S1, dissolving gelatin in water to obtain a gelatin solution;
S2, adding a diamine C₆₋₁₀ alkane into the gelatin solution and mixing evenly by stirring to obtain a mixed solution;
S3, adding a condensing agent into the mixed solution obtained in step S2 to allow a temperature-controlled reaction to obtain a reaction solution; and
S4, dialyzing the reaction solution obtained in step S3 and freeze-drying to obtain the diamine C₆₋₁₀ alkane-modified gelatin.

5. The polyamine-modified gelatin-based hydrogel according to claim 4, wherein the diamine C₆₋₁₀ alkane is selected from the group consisting of hexanediamine, heptanediamine, octanediamine, nonanediamine, and decanediamine.

6. The polyamine-modified gelatin-based hydrogel according to claim 4, wherein a molar ratio of the diamine C₆₋₁₀ alkane to the condensing agent is in a range of (8-12):1.

7. The polyamine-modified gelatin-based hydrogel according to claim 4, wherein the condensing agent is 4-(4,6-dimethoxytriazine-2-yl)-4-methylmorpholine hydrochloride (DMTMM).

8. The polyamine-modified gelatin-based hydrogel according to claim 4, wherein the temperature-controlled reaction in step S3 is conducted at 25° C. to 37° C. for 36 h to 60 h.

9. The polyamine-modified gelatin-based hydrogel according to claim 4, wherein the dialysis process in step S4 is as follows: dialyzing with a 1,000 Da dialysis bag in deionized water for 36 h to 60 h.

10. The polyamine-modified gelatin-based hydrogel according to claim 1, wherein the CHO-PEG derivative is a benzaldehyde-terminated star-shaped multi-arm PEG, and the number of arms of the benzaldehyde-terminated star-shaped multi-arm PEG is in a range of 4 to 8.

11. A method for encapsulating and sustainably releasing a drug, comprising using the polyamine-modified gelatin-based hydrogel according to claim 1.

12. The according to claim 11, wherein the step of encapsulating the drug is as follow: dissolving the drug in the CHO-PEG derivative solution, and then mixing an obtained mixture with the amine-modified gelatin solution to obtain a hydrogel.

13. The method according to claim 12, wherein the CHO-PEG derivative in the CHO-PEG derivative solution has a mass concentration in a range of 10% to 30%; and the amine-modified gelatin in the amine-modified gelatin solution has a mass concentration in a range of 5% to 20%.

14. The method according to claim 12, wherein the amine-modified gelatin in the amine-modified gelatin solution has a mass concentration in a range of 5% to 15%.

15. The method according to claim 12, wherein a concentration of the drug in the mixture obtained by dissolving the drug in the CHO-PEG derivative solution is in the range of 0.5 mg/mL to 1.5 mg/mL.

16. The method according to claim 12, wherein a solvent of the amine-modified gelatin solution is a 0.03 M borax buffer.

17. The method according to claim 12, wherein a solvent of the CHO-PEG derivative solution is a 0.2 M sodium phosphate monobasic buffer.

18. The method according to claim 12, wherein the hydrogel is obtained by mixing the CHO-PEG derivative solution containing the drug and the amine-modified gelatin solution at a volume ratio of 1:1.

19. The method according to claim 11, wherein the drug is a hydrophobic drug selected from the group consisting of epirubicin, indomethacin, irinotecan, and derivatives thereof.
